(19) United States
(12) Patent Application Publication
(10) Pub. No.: US 2004/0030179 A1
(43) Pub. Date: Feb. 12, 2004

(54) Method for producing (meth)acrylic acid esters

(76) Inventors: Friedrich-Georg Martin, Heidelberg (DE); Gerhard Nestler, Ludwigshafen (DE); Jurgen Schroder, Ludwigshafen (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/433,614

(22) PCT Filed: Dec. 17, 2001
(86) PCT No.: PCT/EP01/14903

(30) Foreign Application Priority Data
Dec. 18, 2000 (DE) 100 63 176.2

(51) Int. Cl. C07C 69/52

(52) U.S. Cl. 560/205; 560/224

(57) ABSTRACT

The invention relates to a method for producing (meth)acrylic acid esters by transesterification of the (meth)acrylic acid with an alkanol in the presence of an acid catalyst, a polymerization inhibitor and an organic solvent that forms an azeotropic mixture with water. The reaction mixture is heated to the boiling point of the mixture in a reactor that comprises a distillation unit with a column and a condenser. The azeotropic mixture is distilled off, the organic solvent is returned to the column as reflux and at least a part of the column reflux is contacted with copper or a copper-containing material. The inventive method is advantageous in that substantially no polymerizate is produced in the distillation column.

The present invention relates to a process for the preparation of (meth)acrylates by esterifying (meth)acrylic acid with an alkanol.

(Meth)acrylates are useful starting compounds for the preparation of polymers and copolymers which are used, for example, as surface coatings, dispersions or adhesives. The preparation of (meth)acrylate by acid-catalyzed esterification of (meth)acrylic acid with alkanols is generally known, cf. for example Ullmann's Encyclopedia of Industrial Chemistry, Vol. Al, 162-169, VCH 1985. The formation of the ester from (meth)acrylic acid and alkanol is known to be based on an equilibrium reaction. In order to obtain economical conversions, as a rule one starting material is used in excess and/or the resulting water of esterification is removed from the equilibrium. In order to accelerate and to facilitate the removal of the water, an organic solvent which is immiscible with water or forms an azeotropic mixture with water is generally added. Frequently used solvents are aliphatic, cycloaliphatic and/or aromatic hydrocarbons, such as pentanes, hexanes, heptanes, cyclohexane or toluene, cf. for example DE 20 50 678 A, DE 29 13 218 A, U.S. Pat. No. 4,053,504 A, U.S. Pat. No. 2,917,538 A and EP 618 187 A.

A major problem in the esterification of (meth)acrylic acid is the high tendency of the (meth)acrylate compounds to polymerize owing to their reactive double bonds. This is true in particular if the (meth)acrylate compounds are exposed to relatively high temperatures, cf. for example WO 97/37962. In the preparation and the distillative purification, the (meth)acrylate compounds are exposed to temperatures which can readily trigger undesired polymerization and can lead to polymer formation. This results in soiling of the apparatuses, blockage of pipes and pumps and coating of column trays and heat exchanger surfaces (fouling). The cleaning of the plants is a complicated, expensive and environmentally polluting procedure, cf. DE 10 67 806 A. In addition, the yield and the availability of the plants are greatly reduced thereby.

As a rule, polymerization inhibitors, i.e. compounds which are capable of substantially suppressing the free radical polymerization, are therefore added for stabilization. For the use of the (meth)acrylate compounds, however, the polymerization inhibitors must be separated off, for example by distillation of the (meth)acrylate compounds. In the preparation of high-boiling (meth)acrylate compounds which cannot be purified by distillation, it is possible to use only inhibitors which can be separated off by another method, for example by extraction, filtration or adsorption, or which do not interfere with the further processing. Thus, DE 28 38 691 A describes the use of Cu(I) oxide as an inhibitor, the Cu(I) oxide being removed by extraction. WO 90/07487 describes the use of hydroquinone with the addition of active carbon to the esterification mixture. The active carbon, which is filtered off after the esterification, is added during the esterification in order to avoid discoloration of the ester caused by the hydroquinone. DE 29 13 218 A discloses the use of phosphites, such as triethyl phosphite, as a polymerization inhibitor.

The esterification of (meth)acrylic acid is carried out in general in a reactor to which a distillation column with condenser is attached, said column serving to remove from the reactor the water in the form of the azeotropic mixture with the solvent. The disadvantage of the known processes is that, in spite of the use of polymerization inhibitors, polymer is very rapidly formed in the distillation column attached to the reactor. The run time of the esterification apparatus is thus greatly shortened and complicated and environmentally polluting cleaning work, for example boiling with aqueous alkali solution, is necessary.

It is an object of the present invention to provide a process for the preparation of (meth)acrylates, in particular higher (meth)acrylates, in which the polymer formation in the distillation column attached to the reactor is suppressed.

We have found that this object is achieved and that, surprisingly, the polymer formation can be substantially prevented if at least some of the column reflux is brought into contact with copper or copper-containing materials.

The present invention therefore relates to a process for the preparation of (meth)acrylates by esterifying (meth)acrylic acid with an alkanol in the presence of an acidic catalyst, of a polymerization inhibitor and of an organic solvent which forms an azeotropic mixture with water, with heating in a reactor having a distillation unit, which comprises a column and a condenser, to the boiling point of the reaction mixture, wherein the azeotropic mixture is distilled off, the organic solvent is recycled as reflux to the column and at least some of the column reflux is brought into contact with copper or with copper-containing materials.

In the novel process, the esterification is carried out in a conventional manner. It is effected in one or more reactors connected in series, thorough mixing of the reaction mixture being carried out in a conventional manner, for example by stirring, circulation by means of a pump or natural circulation. Heat can be supplied by heating the wall and/or by means of external or internal heat exchangers, for example tubular or plate-type heat exchangers. Suitable reactors, such as stirred kettles, etc. are known to a person skilled in the art.

In order to carry out the esterification, the starting materials are introduced into the reactor and mixed. The reaction mixture is heated to the boil, and the water formed during the esterification is distilled off as an azeotropic mixture with the organic solvent. This is effected via a distillation unit which is attached to the reactor and comprises a distillation column and a condenser. Distillation columns of conventional design which have internals having a separation effect, for example bubble, sieve or dual-flow trays, or contain dumped or stacked packings are used. A distillation column having dumped packing is preferably used. The packing elements may be of conventional design, for example Raschig, Intos or Pall rings, barrel or Intalox saddles, Top-Pak, etc., cf. also Ullmann's Encyclopedia of Industrial Chemistry, Vol. B3, 4-71 to 4-84, VCH 1988.

The condensers are likewise of known design and may be, for example, tubular or plate-type heat exchangers. They are preferably operated using water or brine.

The azeotropic mixture of the resulting water and the organic solvent is separated off via the distillation column and then condensed in the condenser, the condensate separating into an aqueous phase and an organic phase. The aqueous phase is at least partly discharged or can be further processed to recover the (meth)acrylic acid contained therein. The organic phase is applied at least partly as reflux to the internals of the column or to the column packing.

According to the invention, at least some of the column reflux is brought into contact with copper (copper metal) or with a copper-containing material. Copper-containing materials which can be used are in particular alloys of copper with zinc, tin, aluminum or nickel. However, copper is preferably used (below, reference is always made by way of example to copper alone).

In order to bring the column reflux into contact with copper, it is expedient if the reflux pipe from the condenser to the distillation column is made of copper or is provided with a copper surface, for example by cladding with copper. The walls of the distillation column, in particular in the upper region, can also be provided with a copper surface or with copper internals (which have no separation effect). However, the internals having a separation effect or the packings are preferably at least partly made of copper or provided with a copper surface. It is generally sufficient if from about 1 to 30% of the internals or of the dumped or stacked packing have a copper surface, expediently in the upper region of the column.

The novel process is suitable for the preparation of esters of (meth)acrylic acid with all conventional alkanols. However, it can preferably be used for the preparation of higher (meth)acrylates, in particular those (meth)acrylates which have a molecular weight of >200. Such esters cannot be purified by distillation. Alkanols which may be used are monoalcohols and polyalcohols. The following alcohols are preferably used:

C₈-C₂₀-monoalcohols, such as 2-ethylhexyl, 2-propylheptyl, lauryl or stearyl alcohol;

C₁-C₄-alkyl-substituted cyclopentanols and cyclohexanols, such as tert-butylcyclohexanol;

C₂-C₁₂-diols, such as ethylene glycol, 1,2- and 1,3-propylene glycol, 1,2-, 1,3- or 1,4-butylene glycol, 1,6-hexanediol, etc., and their mono-C₁-C₄-alkyl ethers;

polyethylene glycols and polypropylene glycols, such as diethylene glycol, triethylene glycol, tetraethylene glycol, dipropylene glycol, tripropylene glycol, etc., and their mono-C₁-C₄-alkyl ethers;

triols and higher polyols, such as glycerol, trimethylolpropane, pentaerythritol, etc., or the C₁-C₄-alkyl ethers thereof having at least one free hydroxyl group;

cyclic trimethylolpropane formal (5-ethyl-5-hydroxymethyl-1,3-dioxolane)

the ethoxylated and/or propoxylated derivatives of said alcohols.

The ratio of the number of equivalents of alkanol to the number of equivalents of (meth)acrylic acid is in general from 1:0.7 to 1:2.

A preferably used acidic esterification catalyst is p-toluenesulfonic acid. Other esterification catalysts which may be used are organic sulfonic acids, e.g. methanesulfonic acid, benzenesulfonic acid or dodecylbenzenesulfonic acid, and/or sulfuric acid, which is preferred. The esterification catalyst is used in general in an amount of from 0.1 to 10, preferably from 0.5 to 5, % by weight, based on (meth)acrylic acid and alkanol.

The polymerization inhibitors used are conventional inhibitors, such as hydroquinone, hydroquinone monomethyl ether, 2,6-di-tert-butyl-4-methylphenol, 2,4-di-tert-butyl-6-methylphenol, tert-butylpyrocatechol, p-benzoquinone, p-nitrosophenol, phenothiazine or N-oxyl compounds, such as 4-hydroxy-2,2,6,6-tetramethyl-1-oxylpiperidine, or mixtures thereof. The inhibitors are used as a rule in an amount of from 200 to 2 000 ppm, based on (meth)acrylic acid and alkanol. They can be used with the addition of air or oxygen-containing gas mixtures if required.

Suitable organic solvents are those which form an azeotropic mixture with water. Aliphatic, cycloaliphatic and/or aromatic hydrocarbons, such as pentanes, hexanes, heptanes, cyclohexane or toluene, are preferably used. The solvent is used in general in an amount of from 5 to 50% by weight, based on the reaction mixture.

The esterification is carried out at elevated temperature. The reaction temperature is in general from 60 to 160° C., preferably from 80 to 130° C. The reaction time is in general from 1 to 20, preferably from 2 to 10, hours. The pressure is not critical and it is possible to use reduced, superatmospheric or preferably ambient pressure.

After the esterification, the reaction mixture is expediently subjected to extraction with water and/or with an aqueous alkali or alkaline earth solution. The organic solvent is then distilled off via a distillation column. The (meth)acrylate, together with the catalyst and the inhibitor, remains as a residue and can be further purified, as described, for example, in Ullmann's Encyclopedia of Industrial Chemistry, Vol. Al, 168-169, VCH 1985. For example, the acidic esterification catalyst, residual (meth)acrylic acid and any inhibitor can be removed by extraction with water. The crude ester can then be subjected to steam stripping to remove residual solvent.

The novel process has the advantage that substantially no polymer forms in the distillation column. The conversion is in general from 95 to 98% and the ester content is in general from 90 to 99% by weight. The novel process can be used not only for the preparation of (meth)acrylates but also for the preparation of esters of other α,β-ethylenically unsaturated carboxylic acids, such as crotonic acid, itaconic acid, maleic acid, fumaric acid or citraconic acid, with alkanols and in particular the abovementioned alkanols.

The examples which follow illustrate the invention without restricting it.

### EXAMPLE 1

In a 10 l stirred reactor having double-wall heating and attached distillation column (5 cm×60 cm), a mixture of 2 380 parts of acrylic acid, 2 880 parts of tripropylene glycol, 2 300 parts of cyclohexane, 120 parts of p-toluenesulfonic acid, 9.4 parts of a 50% strength phosphinic acid and 4.7 parts of hydroquinone monomethyl ether was heated to the boil. The resulting water of reaction was discharged as an azeotropic mixture with cyclohexane and condensed, the condensate separating into two phases. The aqueous phase was separated off and the cyclohexane phase was applied as a reflux to the column packing. The column was packed with Raschig rings, the uppermost part of the packing (1 cm) consisting of copper Raschig rings (5 mm) and the remaining part consisting of glass Raschig rings (8 mm). 546 parts of water were separated off in the course of 8 hours (acrylic acid content 3.1%). In accordance with the amount of water of esterification discharged, the conversion was about 97%. The column was free of polymer.

### EXAMPLE 2

The procedure was as in example 1. The distillation column contained only glass Raschig rings (8 mm). After 5 hours, a substantial polymer formation was observed in the column.

### EXAMPLE 3

In a 10 l stirred reactor having double-wall heating and attached distillation column (5 cm×80 cm), a mixture of 2 380 parts of acrylic acid, 2 660 parts of ethoxylated trimethylolpropane (M266), 2 500 parts of cyclohexane, 140 parts of p-toluenesulfonic acid, 10 parts of a 50% strength phosphinic acid and 5 parts of hydroquinone monomethyl ether was heated to the boil. The resulting water of reaction was discharged as an azeotropic mixture with cyclohexane and condensed, the condensate separating into two phases. The aqueous phase was separated off and the cyclohexane phase was applied as a reflux to the column packing. The column was packed with Raschig rings, the uppermost part of the packing (about 1 cm) consisting of copper Raschig rings (5 mm) and the remaining part consisting of glass Raschig rings (8 mm). 538 parts of water were separated off in the course of 9 hours (acrylic acid content 3.5%). In accordance with the amount of water discharged, the conversion was about 95%. The column was free of polymer.

### EXAMPLE 4

The procedure was as in example 3. The distillation column contained only glass Raschig rings (8 mm). After 5 hours, the esterification had to be stopped owing to backing up of liquid in the column because of polymer formation.

We claim:

1. A process for the preparation of (meth)acrylates by esterifying (meth)acrylic acid with an alkanol in the presence of at least one acidic catalyst, at least one polymerization inhibitor and an organic solvent which forms an azeotropic mixture with water, with heating in a reactor having a distillation unit, which comprises a column and a condenser, to the boiling point of the reaction mixture, wherein the azeotropic mixture is distilled off, the organic solvent is recycled as reflux to the column and at least some of the column reflux is brought into contact with copper or with a copper-containing material.

2. A process as claimed in claim 1, wherein at least some of the internals having a separation effect or of the packing of the distillation column consist of copper or of a copper-containing material or are coated therewith.

3. A process as claimed in claim 1 or 2, wherein a packed column which contains copper packings in the upper region is used.

4. A process as claimed in any of the preceding claims, wherein an alkanol which gives a (meth)acrylate having a molecular weight of >200 is used.

5. A process as claimed in claim 4, wherein the alkanol is selected from lauryl alcohol, stearyl alcohol, tert-butylcyclohexanol, ethyldiglycol, cyclic trimethylolpropane formal, 1,6-hexanediol, a polyethylene or polypropylene glycol or a C₁-C₄-monoalkyl ether thereof, glycerol, trimethylolpropane, pentaerythritol and the ethoxylated and/or propoxylated compounds thereof.
